(19) United States
(12) Patent Application Publication
(10) Pub. No.: US 2012/0128778 A1
(43) Pub. Date: May 24, 2012

(54) COMPOSITIONS AND METHODS FOR ONCE-DAILY ADMINISTRATION OF A TRILAYER OSMOTIC TABLET

(76) Inventors: Nipun Davar, Pleasanton, CA (US); Sangita Ghosh, Foster City, CA (US)

(21) Appl. No.: 13/303,623

(22) Filed: Nov. 23, 2011

Related U.S. Application Data
(60) Provisional application No. 61/416,698, filed on Nov. 23, 2010.

(51) Int. Cl. A61K 9/14; A61K 31/4535; A61K 31/5517; A61K 31/47; A61P 1/04; A61K 31/426; A61P 25/08; A61P 25/18; A61P 11/06; A61K 31/357; A61K 31/56

(52) U.S. Cl. 424/490; 514/455; 514/326; 514/220; 514/311; 514/171; 514/370

(57) ABSTRACT

A trilayer osmotic tablet is described. The tablet includes a core, a semi-permeable membrane disposed generally around the core, and an orifice in the semi-permeable membrane in fluid communication with the core. The core includes first, second, and third layers. The first layer is in fluid communication with the orifice. The second layer includes a therapeutically effective dose of a drug and is located adjacent to the first layer. The third layer is located adjacent the second layer. The tablet may include a coating having therapeutically effective doses of at least one additional drug disposed in the coating that generally surrounds the semi-permeable membrane. The at least one additional drug in the coating may be the same or different from the drug in the trilayer core. Methods for treating epilepsy, psychiatric disorders, asthma, and peptic ulcer disease by administering these compositions are also described.

### RELATED APPLICATIONS

This application claims the benefit of U.S. Provisional Application Ser. No. 61/416,698, entitled “Tri-Layer Tablet Formulation for Bi-Modal Delivery,” filed Nov. 23, 2010, which is hereby expressly incorporated by reference in its entirety.

### BACKGROUND OF THE INVENTION

Suprachiasmatic nuclei (SCN) of the anterior hypothalamus, in human brain, influences biological processes in a circadian manner as a pacemaker controls heart rate in a patient with compromised heart function. See Ohdo, S. “Chronotherapeutic strategy: Rhythm monitoring, manipulation, and disruption.” ADVANCED DRUG DELIVERY REVIEWS 62: 859-875 (2010). The current advances in understanding of the influence of circadian rhythm on biological processes have led to important decisions in pharmacotherapy of selected diseases. This understanding strongly suggests that ‘the one size fits all at all times’ approach to drug delivery is no longer optimum. There is a critical need for design and evaluation of robust, spatially, and temporally-controlled drug delivery systems. See Youan, B. C. “Chronopharmaceutical drug delivery systems: Hurdles, hype or hope?” ADVANCED DRUG DELIVERY REVIEWS 62: 898-903 (2010).

### SUMMARY OF THE INVENTION

It has been established that certain physiological functions and diseases follow the circadian rhythm. The effectiveness of a therapeutic substance will be enhanced by aligning its delivery with the time of the day when the symptoms of the disease are most prevalent. FIG. 1 displays, in the form of a 24 hour clock, when symptoms or events of certain diseases are most frequent. Examples of such diseases include but are not limited to epileptic seizures, pain, peptic ulcer, congestive heart failure, asthma, apnea, migraine, rheumatoid arthritis and diabetes. See Ohdo, S. “Chronotherapeutic strategy: Rhythm monitoring, manipulation, and disruption.” ADVANCED DRUG DELIVERY REVIEWS 62: 859-875 (2010)

In one embodiment, the objective of the invention is to provide a trilayer tablet dosage form with the ability to synchronize the delivery of the therapeutic substance(s) with the times of the day when the symptoms are most prevalent.

In another embodiment, the invention provides a trilayer tablet dosage form that delivers dose(s) of a therapeutic substance(s) in two portions, one portion being delivered at night (e.g., bedtime) and the other portion being delivered in the morning (e.g., when waking up).

In another embodiment, the invention provides a trilayer tablet dosage form that will allow for a substantial and predetermined lag time between administration of the tablet and appearance of maximum drug concentration in the body.

In another embodiment, the invention provides a trilayer tablet dosage form that allows for a substantial lag between the two portions of the dose of therapeutic substances.

In another embodiment, the invention provides pharmaceutical compositions having a core, a semi-permeable membrane disposed generally around the core, an orifice in the semi-permeable membrane in fluid communication with the core, and a coating having a first therapeutically effective dose of a first active pharmaceutical ingredient or a pharmaceutically acceptable salt thereof disposed generally around the semi-permeable membrane. The core includes first, second, and third layers. The first layer is in fluid communication with the orifice. The second layer includes a second therapeutically effective dose of a second active pharmaceutical ingredient or a pharmaceutically acceptable salt thereof and is located adjacent to the first layer. The third layer is located adjacent the second layer. The first layer may have a higher viscosity than the second layer.

In another embodiment, the invention provides pharmaceutical compositions having a core, a semi-permeable membrane disposed generally around the core, and an orifice in the semi-permeable membrane in fluid communication with the core. The core includes first, second, and third layers. The first layer is in fluid communication with the orifice. The second layer includes a therapeutically effective dose of an active pharmaceutical ingredient or a pharmaceutically acceptable salt thereof and is located adjacent to the first layer. The third layer is located adjacent the second layer. The first layer may have a higher viscosity than the second layer.

In another embodiment, the invention provides pharmaceutical compositions having a core, a semi-permeable membrane disposed generally around the core, an orifice in the semi-permeable membrane in fluid communication with the core, and a coating having therapeutically effective doses of first and second active pharmaceutical ingredients or pharmaceutically acceptable salts thereof disposed generally around the semi-permeable membrane. The core includes first, second, and third layers. The first layer is in fluid communication with the orifice. The second layer includes a therapeutically effective dose of a third active pharmaceutical ingredient or a pharmaceutically acceptable salt thereof and is located adjacent to the first layer. The third layer is located adjacent the second layer. The first, second, and third active pharmaceutical ingredients may be the same or different.

In another embodiment, the invention provides methods for treating epilepsy. A dosage form is provided and administered to a patient suffering from epilepsy. The dosage form includes a core, a semi-permeable membrane disposed generally around the core, an orifice in the semi-permeable membrane in fluid communication with the core, and a coating that includes a first therapeutically effective dose of a first active pharmaceutical ingredient or a pharmaceutically acceptable salt thereof disposed generally around the semi-permeable membrane. The core includes first, second, and third layers. The first layer is in fluid communication with the orifice. The second layer includes a second therapeutically effective dose of a second active pharmaceutical ingredient or pharmaceutically acceptable salt thereof and is located adjacent to the first layer. The third layer is located adjacent the second layer. A maximum peak plasma concentration resulting from the second therapeutically effective dose occurs within about 5 hours to about 11 hours after the occurrence of a maximum peak plasma concentration resulting from the first therapeutically effective dose.

Active pharmaceutical ingredients that can be used to treat epilepsy include but are not limited to carbamazepine, ethosuximide, oxcarbazepine, phenobarbital, phenytoin, primidone, topiramate, valproate, valproic acid, valproex sodium, felbamate, gabapentin, lamotrigine, levetiracetam, lacosamide, pregabalin, primidone, rufinamide, tiagabine, and zonisamide.

In another embodiment, the invention provides methods for treating a psychiatric disorder (e.g., generalized anxiety disorder). A dosage form is provided and administered to a patient suffering from the psychiatric disorder. The dosage form includes a core, a semi-permeable membrane disposed generally around the core, an orifice in the semi-permeable membrane in fluid communication with the core, and a coating that includes a first therapeutically effective dose of a first active pharmaceutical ingredient or a pharmaceutically acceptable salt thereof disposed generally around the semi-permeable membrane. The core includes first, second, and third layers. The first layer is in fluid communication with the orifice. The second layer includes a second therapeutically effective dose of a second active pharmaceutical ingredient or pharmaceutically acceptable salt thereof and is located adjacent to the first layer. The third layer is located adjacent the second layer. A maximum peak plasma concentration resulting from the second therapeutically effective dose occurs within about 7 hours to about 14 hours after the occurrence of a maximum peak plasma concentration resulting from the first therapeutically effective dose.

Active pharmaceutical ingredients that can be used to treat psychiatric disorders include but are not limited to alprazolam, bretazenil, bromazepam, chlordiazepoxide, cinolazepam, clonazepam, cloxazolam, clorazepate, diazepam, estazolam, fludiazepam, flunitrazepam, flurazepam, halazepam, flutoprazepam, ketazolam, loprazolam, lorazepam, lormetazepam, medazepam, nitrazepam, midazolam, nordazepam, oxazepam, phenazepam, pinazepam, prazepam, quezepam, temazepam, tetrazepam, and triazolam.

In another embodiment, the invention provides methods for treating asthma. A dosage form is provided and administered to a patient suffering from asthma. The dosage form includes a core, a semi-permeable membrane disposed generally around the core, and an orifice in the semi-permeable membrane in fluid communication with the core. The core includes first, second, and third layers. The first layer is in fluid communication with the orifice. The second layer includes a therapeutically effective dose of an active pharmaceutical ingredient or pharmaceutically acceptable salt thereof and is located adjacent to the first layer. The third layer is located adjacent the second layer. A maximum peak plasma concentration resulting from the therapeutically effective dose occurs at least about 5 to about 10 hours after administration of the dosage form.

In another embodiment, the invention provides methods for treating asthma using a combination therapy. A dosage form is provided and administered to a patient suffering from asthma. The dosage form includes a core, a semi-permeable membrane disposed generally around the core, an orifice in the semi-permeable membrane in fluid communication with the core, and a coating that includes therapeutically effective doses of first and second active pharmaceutical ingredients or pharmaceutically acceptable salts thereof disposed generally around the semi-permeable membrane. The core includes first, second, and third layers. The first layer is in fluid communication with the orifice. The second layer includes a therapeutically effective dose of the second active pharmaceutical ingredient or pharmaceutically acceptable salt thereof and is located adjacent to the first layer. The third layer is located adjacent the second layer. The first active pharmaceutical ingredient may be a long acting bronchodilator and the second active pharmaceutical ingredient may be an anti-inflammatory drug.

In another embodiment, the invention provides methods for treating asthma using a combination therapy. A dosage form is provided and administered to a patient suffering from asthma. The dosage form includes a core, a semi-permeable membrane disposed generally around the core, an orifice in the semi-permeable membrane in fluid communication with the core, and a coating that includes therapeutically effective doses of first and second active pharmaceutical ingredients or pharmaceutically acceptable salts thereof disposed generally around the semi-permeable membrane. The core includes first, second, and third layers. The first layer is in fluid communication with the orifice. The second layer includes a therapeutically effective dose of a third active pharmaceutical ingredient or pharmaceutically acceptable salt thereof and is located adjacent to the first layer. The third layer is located adjacent the second layer. The first active pharmaceutical ingredient may be a long acting bronchodilator and the second active pharmaceutical ingredient may be an anti-inflammatory drug. The first, second, and third active pharmaceutical ingredients may be the same or different drugs. In one embodiment, the first active pharmaceutical ingredient is a long acting bronchodilator and the second and third active pharmaceutical ingredients are anti-inflammatory drugs. In the same or another embodiment, the second and third active pharmaceutical ingredients are the same drug.

Active pharmaceutical ingredients that can be used to treat asthma include but are not limited to fluticasone, salmeterol, budesonide, ipratropium, zafirlukast, montelukast, flunisonide, mometasone, prednisolone, beclomethasone, levalbuterol, ciclesonide, formoterol, albuterol, and salbutamol. Long acting bronchodilators include, but are not limited to, salmeterol, formoterol, albuterol, levalbuterol, and salbutamol. Anti-inflammatory drugs include, but are not limited to, fluticasone, budesonide, flunisonide, mometasone, prednisolone, beclomethasone, and ciclesonide. Combinations include, but are not limited to, fluticasone and salmeterol, budesonide and formoterol, mometasone furoate and formoterol fumarate dehydrate, and ipratropium and salbutamol.

In another embodiment, the invention provides methods for treating peptic ulcer disease. A dosage form is provided and administered to a patient suffering from peptic ulcer disease. The dosage form includes a core, a semi-permeable membrane disposed generally around the core, and an orifice in the semi-permeable membrane in fluid communication with the core. The core includes first, second, and third layers. The first layer is in fluid communication with the orifice. The second layer includes a therapeutically effective dose of an active pharmaceutical ingredient or pharmaceutically acceptable salt thereof and is located adjacent to the first layer. The third layer is located adjacent the second layer. A maximum peak plasma concentration resulting from the therapeutically effective dose occurs at least about 1 hour after administration of the dosage form.

Active pharmaceutical ingredients that can be used to treat peptic ulcer disease include but are not limited to famotidine, chlordiazepoxide, methscopolamine, propanthelene, clidinium, pantoprazole, and glycopyrolate.

For any of the dosage forms, pharmaceutical compositions, and methods of this invention, the active pharmaceutical ingredient(s) (drug(s)) contained in the coating may be the same or different than the active pharmaceutical ingredient(s) (drug(s)) contained in the second layer of the trilayer core. Where the active pharmaceutical ingredients (drugs) in the coating and in the second layer are the same, the therapeutically effective doses of the active pharmaceutical ingredient may be the same or different amounts of the drug.

For any of the dosage forms, pharmaceutical compositions, and methods of this invention, the first layer of the trilayer core may have a higher viscosity than the second layer. The first layer may have a viscosity between about 50 and about 20, 000 cps, alternatively between about 50 and about 15,000 cps, alternatively between about 50 and about 10,000 cps, alternatively between about 50 and about 5,000 cps, alternatively between about 50 and about 2,500 cps, alternatively between about 50 and about 1,500 cps, alternatively between about 600 and about 1200 cps, alternatively between about 500 and about 1000 cps. The second layer of the trilayer core may have a viscosity between about 5 and about 10, 000 cps, alternatively between about 5 and about 7,500 cps, alternatively between about 5 and about 5,000 cps, alternatively between about 5 and about 2,500 cps, alternatively between about 5 and about 1,000 cps, alternatively between about 5 and about 500 cps, alternatively between about 5 and about 100 cps, alternatively between about 30 and about 50 cps, alternatively between about 5 and about 50 cps.

For any of the dosage forms, pharmaceutical compositions, and methods of this invention, each of the first and second layers of the trilayer core may include polyethylene oxide having an average molecular weight and the average molecular weight of the polyethylene oxide in the first layer may be higher than the average molecular weight of the polyethylene oxide in the second layer. The average molecular weight of the polyethylene oxide in the first layer may be about 200K to about 1000K, alternatively about 200K to about 800K, alternatively about 200K, alternatively about 300K, alternatively about 400K, alternatively about 500K, alternatively about 600K, alternatively about 700K, alternatively about 800K, alternatively about 900K, alternatively about 1000K. The average molecular weight of the polyethylene oxide in the second layer may be about 100K to about 600K, alternatively about 100K to about 500K, alternatively about 100K to about 400K, alternatively about 100K to about 300K, alternatively about 100K, alternatively about 200K, alternatively about 300K, alternatively about 400K, alternatively about 500K, alternatively about 600K.

For any of the dosage forms, pharmaceutical compositions, and methods of this invention, the weight of the third layer may be ⅓ or more of the total weight of the trilayer core, alternatively ½ or more of the total weight of the trilayer core. Similarly, the weight of the first or second layer may be ⅓ or more of the total weight of the trilayer core, alternatively ½ or more of the total weight of the trilayer core. Furthermore, each of the first, second, and third layers may each comprise the same excipient, e.g., polyethylene oxide.

For any of the dosage forms, pharmaceutical compositions, and methods described herein, the pharmaceutical composition may also have an in vitro release profile as determined in a USP Type H apparatus with a paddle speed of 50 rpm in water at 37° C. such that about 1 to 50 wt % active pharmaceutical ingredient is released within about 2 hours, alternatively within about 1 hour, alternatively within about 0.5 hours following initiation of the in vitro release test and about 50 to 100 wt % is released between about 6 hours to about 18 hours, alternatively between about 6 hours to about 14 hours, alternatively between about 6 hours to about 10 hours following initiation of the in vitro release test. Alternatively or in addition to, the composition may have an in vitro release profile comprising two pulses of drug release each having a maxima as determined in a USP Type II apparatus with a paddle speed of 50 rpm in water at 37° C., wherein the maxima of the two pulses of drug release are separated by about 1 hour to about 12 hours, alternatively by about 1 hour to about 10 hours, alternatively by about 2 hour to about 8 hours, alternatively by about 2 hour to about 6 hours, alternatively by about 1 hour, alternatively by about 2 hours, alternatively by about 3 hours, alternatively by about 4 hours, alternatively by about 5 hours, alternatively by about 6 hours, alternatively by about 7 hours, alternatively by about 8 hours, alternatively by about 9 hours, alternatively by about 10 hours, alternatively by about 11 hours, alternatively by about 12 hours.

For any of the dosage forms, pharmaceutical compositions, and methods described herein, the first and third layers may be substantially free of active pharmaceutical ingredient. In one embodiment, the active pharmaceutical ingredient contained in the second layer of the core does not leach into the first or third layers of the core during in vitro drug release tests, i.e., the active pharmaceutical ingredient does not migrate and subsequently release from the dosage form due to undesirable mixing of layer contents during the in vitro test. During the dissolution of the first layer, less than 5%, alternatively less than 4%, alternatively less than 3%, alternatively less than 2%, alternatively less than 1% of the total dose of active pharmaceutical ingredient (combined amounts from the coating and second layer) is released. Similarly, less than 5%, alternatively less than 4%, alternatively less than 3%, alternatively less than 2%, alternatively less than 1% of the total dose of active pharmaceutical ingredient is released between about 2 hours and about 12 hours, alternatively between about 2 hours and about 10 hours, alternatively between about 2 hours and about 8 hours, alternatively between about 2 hours and about 7 hours, alternatively between about 2 hours and about 6 hours following administration of the pharmaceutical composition or dosage form.

### BRIEF DESCRIPTION OF THE FIGURES

FIG. 1 depicts a display in the form of a 24-hour clock diagram, the approximate time, following diurnal activity/nocturnal sleep routine, when the symptoms or events of disease are worst or most frequent.

FIG. 2 shows the cross-section of a trilayer tablet.

### DETAILED DESCRIPTION

### Example 1

### Trilayer Osmotic Tablet

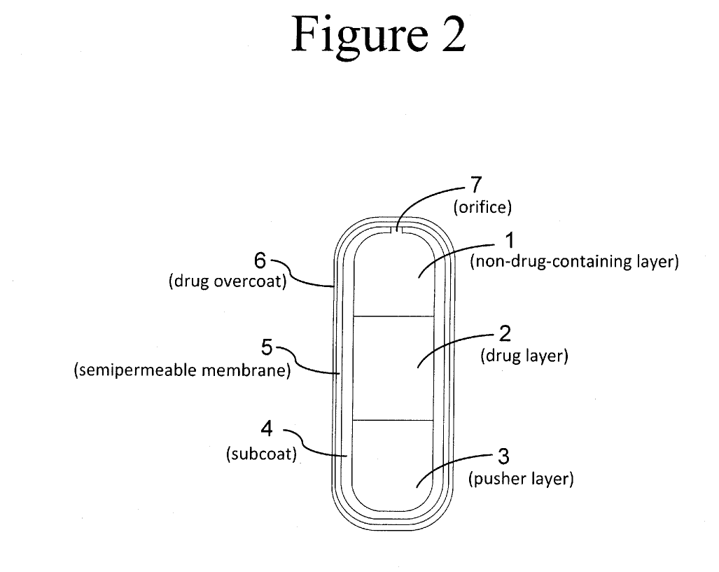

The trilayer osmotic tablet may be in the shape of a capsule that has an outer immediate release coat followed by a semipermeable membrane defining an inner core that contains 3 layers or compartments. As seen in FIG. 2, the tablet contains an inner core with three compartments or layers—first (placebo or non-drug-containing) layer 1, drug-containing layer 2, and pusher layer 3. A semi-permeable membrane 5 having an orifice 7 is generally disposed around, or surrounds, the inner core. An outer drug-containing coat 6 may be generally disposed around, or surrounds, the semi-permeable membrane 5. The tablet may also include a subcoat 4, which is located between the inner core and semi-permeable membrane 5.

For the tablets that include an outer drug-containing coat 6, a first active pharmaceutical ingredient (drug) contained in the outer drug-containing coat 6 may be the same or different as a second active pharmaceutical ingredient (drug) contained in the second layer 2 of the trilayer core. Where the first and second active pharmaceutical ingredients (drugs) are the same, the amount of the first and second active pharmaceutical ingredients (drugs) may be the same or different. The outer drug-containing coat 6 may contain at least one active pharmaceutical ingredient (drug), alternatively at least two active pharmaceutical ingredients (drugs), alternatively at least three active pharmaceutical ingredients (drugs). In one embodiment, at least one of the active pharmaceutical ingredients contained in the drug-containing coat 6 may be the same as an active pharmaceutical ingredient contained in the second layer. Alternatively, the active pharmaceutical ingredient(s) contained in the drug-containing coat 6 may be different than the active pharmaceutical ingredient contained in the second layer.

### The Multi-Layer Tablet

The relative weight percentages (relative to the total weight of the trilayer core) of the first (placebo or non-drug-containing) layer 1, second (drug-containing) layer 2, and third (pusher) layer 3 may be about 25%, about 25%, and about 50%, respectively. Alternatively, the relative weight percentages (relative to the total weight of the trilayer core) of the first layer 1, drug-containing layer 2, and pusher layer may be about 33⅓%, about 33 ⅓%, and about 33⅓%, respectively. Alternatively, the relative weight percentages (relative to the total weight of the trilayer core) of the first layer 1, drug-containing layer 2, and pusher layer 3 may be between about 25% to about 33⅓%, between about 25% to about 33⅓%, and between about 33⅓% to about 50%, respectively.

The capsule-shaped tablet may contain an inner coating 4 that immediately surrounds (or is generally disposed around) the inner core and serves as a flow promoting layer. The inner coating 4 may include hydroxyl propyl methyl cellulose (HPMC), hydoxypropyl cellulose, hydroxyethyl cellulose, or povidone. Inner coat 4 may be composed of between about 5% to about 15%, alternatively about 1% to about 25%, alternatively about 1% to about 20%, alternatively about 5% to about 20% of the tablet core weight.

The capsule-shaped tablet contains semi-permeable membrane 5 that serves as the rate-controlling membrane. The semi-permeable membrane 5 may include cellulose acetate and a pore-forming copolymer such as polypropylene glycol or poloxamer 188. The ratio of cellulose acetate to polyethylene glycol may be about 99:1 to about 95:5. The ratio of cellulose acetate to poloxamer may be about 90:10 to about 80:20. The amount of the semipermeable membrane ranges from about 5 wt % to about 80 wt % of the core tablet

When desired, the outer coat 6 may be included that contains at least one drug that can be immediately released after ingestion of the tablet. The outer coat 6 may contain the same drug or a different drug that is contained in the second layer of the trilayer core. Where the outer coat 6 contains the same drug as the second drug-containing layer 2, the same amount of drug may be contained in the drug-containing layer 2 and the outer coat 6. Alternatively, the outer coat 6 may contain more or less drug that contained in the drug-containing layer 2.

First layer 1 of the inner core is located adjacent to and is in fluid communication with the orifice 7 of the semi-permeable membrane 5. The first layer 1 may contain suspending polymer (e.g., about 50-80% polyethylene oxide), a disintegrant (e.g., about 2-10 wt % of croscarmellose sodium), a binder (e.g., about 2-8 wt % povidone), an osmotic agent (e.g., about 10-30 wt % sodium chloride), and a lubricant (e.g., about 0.1-1 wt % magnesium stearate) (wt % of the placebo layer). The first layer 1 may be substantially free of drug (active pharmaceutical ingredient). It may contain less than 2%, alternatively less than 1%, alternatively less than 0.5%, alternatively less than 0.2%, alternatively less than 0.1%, alternatively less than 0.01% of the active pharmaceutical ingredient.

The drug-containing layer 2 (active pharmaceutical ingredient-containing layer) is located between (and adjacent to) and in contact with the first layer 1 and pusher layer 3. In one embodiment, the drug-containing layer 2 may contain about 0.2 to about 2.4 wt % drug, a disintegrant (e.g., about 2-10% of croscarmellose sodium), an osmotic agent (e.g., about 10-30 wt % of sodium chloride), a binder (e.g., about 2-8 wt % povidone), a glidant (e.g., about 0.5-4 wt % silicon dioxide), a lubricant (e.g., about 0.1-1 wt % magnesium stearate), an antioxidant (e.g., about 0.01-1 wt % of butylated hydroxy toluene), and a suspending polymer (e.g., about 50-80 wt % polyethylene oxide) (wt % of the drug-containing layer).

The expandable pusher layer 3 is located adjacent to and in contact with the drug-containing layer 2. The pusher layer 3 may contain an osmotic agent (e.g., about 10 wt % to about 30 wt % sodium chloride), a binder (e.g., about 2 wt % to about 10 wt % povidone), a glidant (e.g., about 0.5 wt % to about 4 wt % silicon dioxide), a lubricant (e.g., about 0.1 wt % to about 2 wt % magnesium stearate), and a suspending polymer (e.g., about 60 wt % to about 80% polyethylene oxide) (wt % of the total pusher layer). The expandable pusher layer 3 may be substantially free of drug (active pharmaceutical ingredient). It may contain less than 2%, alternatively less than 1%, alternatively less than 0.5%, alternatively less than 0.2%, alternatively less than 0.1%, alternatively less than 0.01% drug (active pharmaceutical ingredient). The expandable pusher layer 3 may account for 33% or more, alternatively 40% or more, alternatively 50% or more, alternatively 60% or more, alternatively 70% or more of the total weight of the trilayer osmotic core.

The polyethylene oxide described above may have average molecular weights between 50K and 9 million. For the placebo layer 1, the polyethylene oxide may have an average molecular weight of about 200K, alternatively about 300K, alternatively about 400K, alternatively about 500K, alternatively about 600K, alternatively about 700K, alternatively about 800K. For the drug-containing layer 2, the polyethylene oxide may have an average molecular weight of about 50K, alternatively about 100K, alternatively about 200K, alternatively about 300K, alternatively about 400K, alternatively about 500K. For the expandable push layer 3, the polyethylene oxide may have an average molecular weight of about 2 million, alternatively about 3 million, alternatively about 4 million, alternatively about 5 million, alternatively about 6 million, alternatively about 7 million, alternatively about 8 million, alternatively about 9 million.

### Example 2

### Treatment of Epilepsy

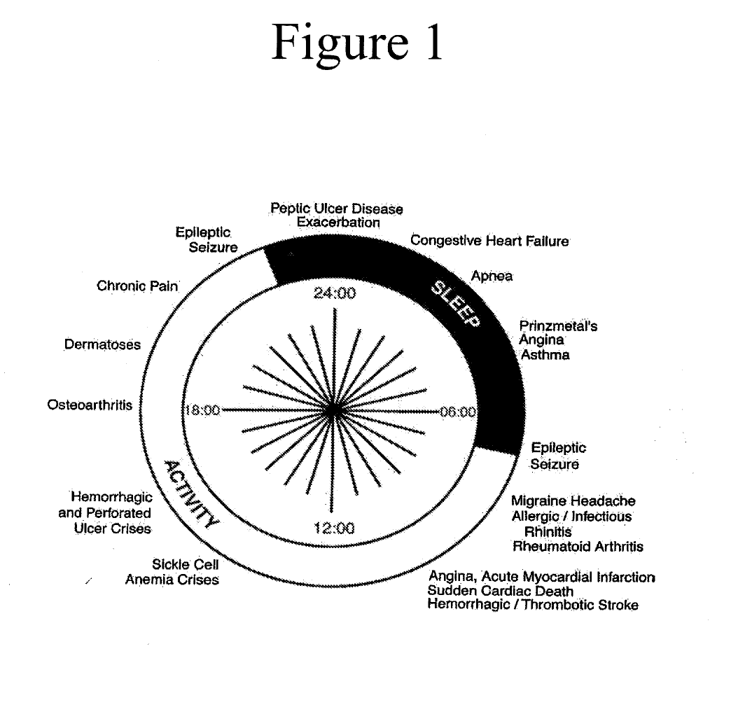

As shown in FIG. 1, epileptic seizures may occur most frequently around about 11 PM, alternatively between about 9 PM and about 1 AM, at night and around about 7 AM, alternatively between about 5 AM and about 9 AM, in the morning. The coated trilayer tablet dosage form could deliver the drug(s) in two portions, such that the maximum plasma concentrations resulting from the delivery/release of the first and second portions of the drug(s) occurs at night (e.g., around about 11 PM) and in the morning (e.g., around about 7 AM) or vice versa. The first portion of the drug(s) (active pharmaceutical ingredient(s)) may be contained in a coating that is generally disposed around the semi-permeable membrane that is disposed generally around the trilayer core. The second portion of the drug(s) could be contained in the second layer (drug layer) of the trilayer core.

The delivery/release of the first portion (first therapeutically effective dose) may result in a first maximum plasma concentration (first Cₘₐₓ, and Tₘₐₓ) around about 11 PM, alternatively around about 9 PM, alternatively around about 9:30 PM, alternatively around about 10 PM, alternatively around about 10:30 PM, alternatively around about 11 PM, alternatively around about 11:30 PM, +/− about 30 minutes at night. Delivery/release of the first portion (first therapeutically effective dose) may also result in a first maximum plasma concentration (first Cₘₐₓ, and Tₘₐₓ) between about 8 PM and about 12 AM, alternatively between about 9 PM and about 12 AM, alternatively between about 10 PM and about 12 AM, alternatively between about 9 PM and about 11 PM, alternatively between about 10 PM and about 11 PM. (Note that all times expressed in this application would apply to the time zone where the patient resides and/or where the dose is administered.)

Delivery/release of the second portion (second therapeutically effective dose) may result in a second maximum plasma concentration (second Cₘₐₓ, and Tₘₐₓ) around about 7 AM, alternatively around about 5 AM, alternatively around about 6 AM, alternatively around about 8 AM, alternatively around about 9 AM, +/− about 30 minutes in the morning. Delivery/release of the second portion (second therapeutically effective dose) may also result in a second maximum plasma concentration (second Cₘₐₓ, and Tₘₐₓ) between about 5 AM and about 9 AM, alternatively between about 6 AM and about 8 AM, alternatively between about 7 AM and about 8 AM. Alternatively, the first portion (first therapeutically effective dose) could be administered in the morning (e.g., about 7 AM) and the second portion (second therapeutically effective dose) could be administered in the evening/night (e.g., about 11 PM). An example of the trilayer tablet dosage form is listed in Table 1 below.

A maximum peak plasma concentration resulting from the second portion (second therapeutically effective dose) may occur within about 5 hours to about 11 hours, alternatively about 5 hours to about 10 hours, alternatively about 6 hours to about 10 hours, alternatively about 7 hours to about 10 hours, alternatively about 6 hours to about 9 hours, alternatively about 6 hours to about 8 hours, after the occurrence of a maximum peak plasma concentration resulting from the first therapeutically effective dose or from the time of administration of the dosage form. Alternatively, the maximum peak plasma concentration resulting from the second portion (second therapeutically effective dose) may occur at least about 5 hours, alternatively about 6 hours, alternatively about 7 hours, alternatively about 8 hours, alternatively about 9 hours, alternatively about 10 hours, alternatively about 11 hours, alternatively about 12 hours, alternatively about 13 hours, alternatively about 14 hours, alternatively about 15 hours after the occurrence of a maximum peak plasma concentration resulting from the first therapeutically effective dose or from the time of administration of the dosage form.

Drugs that could be used to treat epileptic seizures include but are not limited to carbamazepine, ethosuximide, oxcarbazepine, phenobarbital, phenytoin, primidone, topiramate, valproate, valproic acid, valproex sodium, felbamate, gabapentin, lamotrigine, levetiracetam, lacosamide, pregabalin, primidone, rufinamide, tiagabine and zonisamide.

The symptoms associated with epileptic seizures are most frequent at about 10 PM to about 11 PM and at about 7 AM to about 8 AM. The diurnal manifestation of these symptoms could be addressed by using a bimodal release of an agent, such as, topiramate and tiagabine. The compositions for trilayer tablets containing topiramate and tiagabine are listed in Table 1 and Table 2, respectively.

TABLE 1
Composition of Topiramate Trilayer Tablet

| | Amount per |
|---|---|
| Components | Tablet (mg) |
| First Layer | |
| Polyethylene oxide, 300K | 191.175 |
| Povidone (K29-32) | 6.25 |
| Croscarmellose sodium | 7.5 |
| Silicon Dioxide (Sylloid 244FP) | 5 |
| Sodium chloride | 37.5 |
| Magnesium Stearate | 1.25 |
| Butylated hydroxytoluene | 0.075 |
| Ferric oxide | 1.25 |
| Total weight of the first layer | 250 |
| Second Layer | |
| Topiramate | 100 |
| Polyethylene oxide, 100K | 78.6 |
| Povidone (K29-32) | 7.5 |
| Croscarmellose sodium | 15 |
| Silicon Dioxide (Sylloid 244FP) | 3 |
| Sodium chloride | 45 |
| Magnesium Stearate | 0.75 |
| Butylated hydroxytoluene | 0.075 |
| Ferric oxide | 0.075 |
| Total weight of the second layer | 250 |
| Push Layer | |
| Polyethylene oxide, 7000K | 362.25 |
| Povidone (K29-32) | 25 |
| Silicon Dioxide (Sylloid 244FP) | 6.65 |
| Sodium chloride | 100 |
| Magnesium Stearate | 2.5 |
| Butylated hydroxytoluene | 0.25 |
| Ferric oxide | 3.35 |
| Total weight of the push layer | 500 |
| Subcoat | |
| Hydroxypropylmethyl cellulose | 100 |
| Total weight of the subcoat | 100 |
| Rate controlling membrane | |
| Cellulose acetate | 176 |
| Polaxamer F68 | 24 |
| Total weight of the rate controlling | 200 |
| membrane | |
| Immediate release overcoat | |
| Topiramate | 100 |
| Hydroxypropylmethyl cellulose | 100 |
| Total weight of the immediate release | 200 |
| overcoat | |
| Total tablet weight | 1500 |

TABLE 2
Composition of Tiagabine Trilayer Tablet

| | Amount per |
|---|---|
| Components | Tablet (mg) |
| First Layer | |
| Polyethylene oxide, 1 MM | 38.235 |
| Povidone (K29-32) | 1.25 |
| Croscarmellose sodium | 1.5 |
| Silicon Dioxide (Sylloid 244FP) | 1 |
| Sodium chloride | 7.5 |
| Magnesium Stearate | 0.25 |
| Butylated hydroxytoluene | 0.015 |
| Ferric oxide | 0.25 |
| Total weight of the first layer | 50 |
| Second Layer | |
| Tiagabine HCl | 16 |
| Polyethylene oxide, 100K | 30.95 |
| Povidone (K29-32) | 2.5 |
| Croscarmellose sodium | 5 |
| Silicon Dioxide (Sylloid 244FP) | 1 |
| Sodium chloride | 10 |
| Magnesium Stearate | 0.5 |
| Butylated hydroxytoluene | 0.025 |
| Ferric oxide | 0.025 |
| Total weight of the second layer | 66 |
| Push Layer | |
| Polyethylene oxide, 7000K | 72.45 |
| Povidone (K29-32) | 5 |
| Silicon Dioxide (Sylloid 244FP) | 1.33 |
| Sodium chloride | 20 |
| Magnesium Stearate | 0.5 |
| Butylated hydroxytoluene | 0.05 |
| Ferric oxide | 0.67 |
| Total weight of the push layer | 100 |
| Subcoat | |
| Hydroxypropylmethyl cellulose | 10 |
| Total weight of the subcoat | 10 |
| Rate controlling membrane | |
| Cellulose acetate | 18.5 |
| Polaxamer F68 | 3.5 |
| Total weight of the rate controlling | 22 |
| membrane | |
| Immediate release overcoat | |
| Tiagabine HCl | 16 |
| Hydroxypropylmethyl cellulose | 34 |
| Total weight of the immediate release | 50 |
| overcoat | |
| Total tablet weight | 298 |

### Example 3

### Treatment of Psychiatric Disorders

Benzodiazepines are a drug class that are commonly used to treat psychiatric disorders, such as, generalized anxiety disorder. Presently, patients have to take more than one tablet per day to get a sufficient effect. A trilayer tablet composition, as listed in Table 3, could be used to deliver two doses of a drug (e.g., alprazolam) or doses of two different drugs, in a pulsatile manner, over a period of 24 hours for the treatment of psychiatric disorders, such as, generalized anxiety disorder. A pulsatile delivery of drug(s) could provide the initial feeling of ‘drug-on-board’ for the anxious patients, compared to a slow onset of a constant release profile. Furthermore, the trilayer tablet dosage form provides the convenience of once-daily regimen for the patients, which may result in superior compliance.

The delivery/release of the first portion/therapeutically effective dose may result in a first maximum plasma concentration (first Cₘₐₓ, and Tₘₐₓ) within about 4 hours, alternatively about 3 hours, alternatively about 2 hours, alternatively about 1 hour of administration of the dosage form.

Delivery/release of the second portion/therapeutically effective dose may result in a second maximum plasma concentration (second Cₘₐₓ, and Tₘₐₓ) at least about 6 hours, alternatively about 7 hours, alternatively about 8 hours, alternatively about 9 hours, alternatively about 10 hours, alternatively about 11 hours, alternatively about 12 hours, alternatively about 13 hours, alternatively about 14 hours, alternatively about 15 hours after administration of the dosage form. Alternatively, the second portion/therapeutically effective dose may result in a second maximum plasma concentration (second Cₘₐₓ, and Tₘₐₓ) at least about 6 hours, alternatively about 7 hours, alternatively about 8 hours, alternatively about 9 hours, alternatively about 10 hours, alternatively about 11 hours, alternatively about 12 hours, alternatively about 13 hours, alternatively about 14 hours, alternatively about 15 hours after the occurrence of the maximum plasma concentration resulting from the release of the first therapeutically effective dose.

A maximum peak plasma concentration resulting from the second therapeutically effective dose may occur within about 5 hours to about 14 hours, alternatively about 6 hours to about 13 hours, alternatively about 6 hours to about 12 hours, alternatively about 7 hours to about 11 hours, alternatively about 6 hours to about 10 hours, alternatively about 6 hours to about 9 hours, after the occurrence of a maximum peak plasma concentration resulting from the first therapeutically effective dose or from the time of administration of the dosage form. Alternatively, the maximum peak plasma concentration resulting from the second therapeutically effective dose may occur at least about 5 hours, alternatively about 6 hours, alternatively about 7 hours, alternatively about 8 hours, alternatively about 9 hours, alternatively about 10 hours, alternatively about 11 hours, alternatively about 12 hours, alternatively about 13 hours, alternatively about 14 hours, alternatively about 15 hours after the occurrence of a maximum peak plasma concentration resulting from the first therapeutically effective dose or from the time of administration of the dosage form.

Drugs used to treat psychiatric disorders include, but are not limited to, alprazolam, bretazenil, bromazepam, chlordiazepoxide, cinolazepam, clonazepam, cloxazolam, clorazepate, diazepam, estazolam, fludiazepam, flunitrazepam, flurazepam, halazepam, flutoprazepam, ketazolam, loprazolam, lorazepam, lormetazepam, medazepam, nitrazepam, midazolam, nordazepam, oxazepam, phenazepam, pinazepam, prazepam, quezepam, temazepam, tetrazepam, and triazolam. The same drug may be included in both the coating and the second layer of the trilayer core. Alternatively, a first drug may be included in the coating and a different second drug may be included in the second layer of the trilayer core.

A trilayer tablet containing alprazolam for use in the invention is prepared as follows: the first layer, placebo layer, comprises of croscarmellose sodium, povidone (K-29-32), sodium chloride, magnesium stearate, iron oxide color, BHT and polyethylene oxide 1 million average molecular weight. All ingredients are screened separately through a 30 mesh screen then dry blended excluding magnesium stearate in a conventional blended for 13 minutes followed by the addition of magnesium stearate with the blending continued for another 5 minutes to yield a homogenous blend. The second layer (drug layer) contains alprazolam, croscarmellose sodium, butylated hydroxytoluene, sodium chloride, povidone (K-29-32), sylloid 244 FP, magnesium stearate, iron oxide color, and polyethylene oxide 100K average molecular weight. The ingredients are screened and blended identically to the placebo layer described above. The third layer (push layer) includes butylated hydroxytoluene, sodium chloride, povidone (K-29-32), sylloid 244 FP, magnesium stearate, iron oxide color, and polyethylene oxide 7 million average molecular weight. The ingredients are screened and blended identically to the placebo and drug layer described above. The placebo layer (50 mg), drug layer (50 mg), and push layer (100 mg) are sequentially added into a 6/32 inch (4.76 cm) diameter punch-die set and tamped in between each layer. The three layers are compressed under a pressure head of 500 psi using a Carver press.

The trilayer tablet containing alprazolam is then coated with a polymer coating. This subcoat is prepared from a 10% of aqueous solution of hydroxypropylmethylcellulose (HPMC). The solution is sprayed onto the trilayer cores as prepared above to provide a 17.5 mg subcoat layer. The coated tablet with HPMC is dried for 1 hour at 48° C.

Table 3 represents a typical formulation of an alprazolam trilayer tablet. The trilayer tablet is manufactured into a dosage form by being coated with another layer of polymer coating. This second coating layer (semipermeable membrane) is prepared from cellulose acetate and polaxamer, which is dissolved in a cosolvent of acetone and water. The wall-forming composition is sprayed onto and around the HPMC-coated trilayer core to provide a 43.8 mg semipermeable wall. Next, the semipermeable membrane-walled, trilayer tablet is drilled to provide a 20 mil (0.51 mm) orifice such that the placebo and the exterior of the dosage form are in fluid communication via the orifice. The residual solvent is removed by drying for 72 hours at 45° C. and 45% relative humidity. Next, the dosage form is dried further for 6 hours at 45° C. to remove excess moisture. The tablet is then coated with an aqueous solution containing alprazolam and HPMC.

TABLE 3
Composition of Alprazolam Trilayer Tablet

| | Amount per |
|---|---|
| Components | Tablet (mg) |
| First Layer | |
| Polyethylene oxide, 1 MM | 38.235 |
| Povidone (K29-32) | 1.25 |
| Croscarmellose sodium | 1.5 |
| Silicon Dioxide (Sylloid 244FP) | 1 |
| Sodium chloride | 7.5 |
| Magnesium Stearate | 0.25 |
| Butylated hydroxytoluene | 0.015 |
| Ferric oxide | 0.25 |
| Total weight of the first layer | 50 |
| Second Layer | |
| Alprazolam | 0.5 |
| Polyethylene oxide, 100K | 25.7 |
| Povidone (K29-32) | 2.5 |
| Croscarmellose sodium | 5 |
| Silicon Dioxide (Sylloid 244FP) | 1 |
| Sodium chloride | 15 |
| Magnesium Stearate | 0.25 |
| Butylated hydroxytoluene | 0.025 |
| Ferric oxide | 0.025 |
| Total weight of the second layer | 50 |
| Push Layer | |
| Polyethylene oxide, 7000K | 72.45 |
| Povidone (K29-32) | 5 |
| Silicon Dioxide (Sylloid 244FP) | 1.33 |
| Sodium chloride | 20 |
| Magnesium Stearate | 0.5 |
| Butylated hydroxytoluene | 0.05 |
| Ferric oxide | 0.67 |
| Total weight of the push layer | 100 |
| Subcoat | |
| Hydroxypropylmethyl cellulose | 17.5 |
| Total weight of the subcoat | 17.5 |
| Rate controlling membrane | |
| Cellulose acetate | 37.7 |
| Polaxamer F68 | 6.1 |
| Total weight of the rate controlling | 43.8 |
| membrane | |
| Immediate release overcoat | |
| Alprazolam | 0.5 |
| Hydroxypropylmethyl cellulose | 31.68 |
| Total weight of the immediate release | 32.18 |
| overcoat | |
| Total tablet weight | 293.48 |

### Example 4

### Treatment of Asthma

### Example 4a

### Trilayer Tablet

As shown in FIG. 1, patients frequently experience asthma attacks prior to waking up, around about 4 AM. Furthermore, the middle of the night is an inconvenient time for administration of a dosage form. A dosage form is exemplified that will allow for the patient to administer the trilayer tablet prior to bedtime, e.g., around about 9 PM.

A maximum peak plasma concentration resulting from the therapeutically effective dose may occur within about 5 hours to about 14 hours, alternatively about 5 hours to about 13 hours, alternatively about 5 hours to about 12 hours, alternatively about 6 hours to about 13 hours, alternatively about 6 hours to about 12 hours, alternatively about 7 hours to about 11 hours, alternatively about 6 hours to about 10 hours, alternatively about 6 hours to about 9 hours, after the administration of the dosage form. Alternatively, the maximum peak plasma concentration resulting from the therapeutically effective dose may occur at least about 4 hours, alternatively about 5 hours, alternatively about 6 hours, alternatively about 7 hours, alternatively about 8 hours, alternatively about 9 hours, alternatively about 10 hours, alternatively about 11 hours, alternatively about 12 hours, alternatively about 13 hours, alternatively about 14 hours, alternatively about 15 hours, after the administration of the dosage form.

This dosage form may have a sufficient lag time between administration of the trilayer tablet and maximum plasma concentration (of the active drug) such that the maximum plasma concentration coincides with a frequent time of asthmatic attacks early in the morning, e.g. around about 4 AM. The dosage form could be formulated such that there is a lag time of at least about 12 hours, alternatively about 11 hours, alternatively about 10 hours, alternatively about 9 hours, alternatively about 8 hours, alternatively about 7 hours, alternatively about 6 hours, alternatively about 5 hours, alternatively about 4 hours, +/− about 30 minutes between administration/ingestion of the trilayer tablet and the time of the maximum plasma concentration (Cₘₐₓ and Tₘₐₓ) of the drug. Alternatively, the dosage form could be formulated such that there is a lag time of about 4 to about 12 hours, alternatively about 5 to about 11 hours, alternatively about 6 to about 11 hours, alternatively about 6 to about 10 hours, alternatively about 6 to about 9 hours, alternatively about 7 to about 11 hours, alternatively about 10 to about 11 hours, between administration/ingestion of the trilayer tablet and the time of the maximum plasma concentration (Tₘₐₓ) of the drug.

The delivery/release of the therapeutically effective dose of the active pharmaceutical ingredient or pharmaceutically acceptable salt thereof may result in the maximum plasma concentration (Cₘₐₓ, and Tₘₐₓ) occurring around about 4 AM, alternatively around about 1 AM, alternatively around about 2 AM, alternatively around about 3 AM, alternatively around about 5 AM, alternatively around about 6 AM, alternatively around about 7 AM, +/− about 30 minutes. The delivery/release of the therapeutically effective dose of the active pharmaceutical ingredient or pharmaceutically acceptable salt thereof may also result in the maximum plasma concentration (Cₘₐₓ, and Tₘₐₓ) occurring between about 1 AM and about 7 AM, alternatively between about 2 AM and about 6 AM, alternatively between about 3 AM and about 5 AM, alternatively between about 3:30 AM and about 4:30 AM. (Note that all times expressed in this application would apply to the time zone where the patient resides and/or where the dose is administered.)

Drugs that could delivered to treat asthma according to this invention include but are not limited to fluticasone, salmeterol, budesonide, ipratropium, zafirlukast, montelukast, flunisonide, mometasone, prednisolone, beclomethasone, levalbuterol, ciclesonide, formoterol, albuterol, and salbutamol.

An example of a trilayer tablet including montelukast is listed in Table 4.

TABLE 4
Composition of Montelukast Trilayer Tablet

| | Amount per |
|---|---|
| Components | Tablet (mg) |
| First Layer | |
| Polyethylene oxide, 1 MM | 38.235 |
| Povidone (K29-32) | 1.25 |
| Croscarmellose sodium | 1.5 |
| Silicon Dioxide (Sylloid 244FP) | 1 |
| Sodium chloride | 7.5 |
| Magnesium Stearate | 0.25 |
| Butylated hydroxytoluene | 0.015 |
| Ferric oxide | 0.25 |
| Total weight of the first layer | 50 |
| Second Layer | |
| Montelukast sodium | 10 |
| Polyethylene oxide, 100K | 30.95 |
| Povidone (K29-32) | 2.5 |
| Croscarmellose sodium | 5 |
| Silicon Dioxide (Sylloid 244FP) | 1 |
| Sodium chloride | 10 |
| Magnesium Stearate | 0.5 |
| Butylated hydroxytoluene | 0.025 |
| Ferric oxide | 0.025 |
| Total weight of the second layer | 60 |
| Push Layer | |
| Polyethylene oxide, 7000K | 72.45 |
| Povidone (K29-32) | 5 |
| Silicon Dioxide (Sylloid 244FP) | 1.33 |
| Sodium chloride | 20 |
| Magnesium Stearate | 0.5 |
| Butylated hydroxytoluene | 0.05 |
| Ferric oxide | 0.67 |
| Total weight of the push layer | 100 |
| Subcoat | |
| Hydroxypropylmethyl cellulose | 5 |
| Total weight of the subcoat | 5 |
| Rate controlling membrane | |
| Cellulose acetate | 39.6 |
| Polyethylene glycol 3350 | 0.4 |
| Total weight of the rate controlling | 40 |
| membrane | |
| — | |
| Total tablet weight | 255 |

### Example 4b

### Coated Trilayer Tablet—Combination Therapy

Two or more medications used to treat asthma combined into one dosage form may be beneficial for effective asthma treatment. These dosage forms may be referred to as “combination medications.”

Long acting bronchodilators (LABAs) control the symptoms of asthma through bronchodilation. Inflammation of the bronchial tubes associated with asthma also needs to be controlled, both acutely and chronically over long periods of time. If the inflammation is not controlled, it can lead to a severe asthma attack. Thus, in addition to needing an acute dose of a bronchodilator (for the symptom control) and a dose of an anti-inflammatory for acute inflammation control, a patient suffering from asthma also needs an anti inflammatory working throughout the day to treat chronic inflammation. Hence a combination of a bronchodilator and an anti-inflammatory is delivered in the dosage form as described in the present invention to treat asthma. This enables effective treatment of two areas of asthma concurrently: (1) a bronchodilator works by widening the airways, thereby making it easier to breathe, and (2) an anti-inflammatory (e.g., steroid) reduces and prevents chronic inflammation of the airways.

Drugs may be delivered in combination at an appropriate dose to treat asthma according to this invention. Drugs that could delivered to treat asthma according to this invention include but are not limited to fluticasone, salmeterol, budesonide, ipratropium, zafirlukast, montelukast, flunisonide, mometasone, prednisolone, beclomethasone, levalbuterol, ciclesonide, formoterol, albuterol, and salbutamol. In particular, long acting bronchodilators include, but are not limited to, salmeterol, formoterol, albuterol, levalbuterol, and salbutamol. Anti-inflammatory drugs include, but are not limited to, fluticasone, budesonide, flunisonide, mometasone, prednisolone, beclomethasone, and ciclesonide. Combinations include, but are not limited to, fluticasone and salmeterol, budesonide and formoterol, mometasone furoate and formoterol fumarate dehydrate, and ipratropium and salbutamol.

A trilayer tablet as described in the present invention is effectively used to deliver a combination of drugs to treat asthma. A long-acting bronchodilator, e.g., salmeterol and an anti-inflammatory drug, e.g., a corticosteroid such as fluticasone, may be provided to the patient as an immediate release dose, followed by an additional anti-inflammatory drug, e.g., longer acting steroid, to control the inflammation of the airways. The anti-inflammatory drugs in the coating and second layer of the tablet may be the same or different drugs. An example of a trilayer tablet, including a combination of fluticasone and salmeterol, is listed in Table 5.

TABLE 5
Composition of a Trilayer Tablet containing a combination
of fluticasone propionate and salmeterol

| | Amount per |
|---|---|
| Components | tablet (mg) |
| First Layer | |
| Polyethylene oxide, 1 MM | 38.235 |
| Povidone (K29-32) | 1.25 |
| Croscarmellose sodium | 1.5 |
| Silicon Dioxide (Sylloid 244FP) | 1 |
| Sodium chloride | 7.5 |
| Magnesium Stearate | 0.25 |
| Butylated hydroxytoluene | 0.015 |
| Ferric oxide | 0.25 |
| Total weight of the first layer | 50 |
| Second Layer | |
| fluticasone propionate | 0.45 |
| Polyethylene oxide, 100K | 41 |
| Povidone (K29-32) | 2.5 |
| Croscarmellose sodium | 5 |
| Silicon Dioxide (Sylloid 244FP) | 1 |
| Sodium chloride | 10 |
| Magnesium Stearate | 0.5 |
| Butylated hydroxytoluene | 0.025 |
| Ferric oxide | 0.025 |
| Total weight of the second layer | 60.5 |
| Push Layer | |
| Polyethylene oxide, 7000K | 60 |
| Povidone (K29-32) | 5 |
| Silicon Dioxide (Sylloid 244FP) | 1.33 |
| Sodium chloride | 13 |
| Magnesium Stearate | 0.5 |
| Butylated hydroxytoluene | 0.05 |
| Ferric oxide | 0.67 |
| Total weight of the push layer | 80.55 |
| Subcoat | |
| Hydroxypropylmethyl cellulose | 11.5 |
| Total weight of the subcoat | 11.5 |
| Rate controlling membrane | |
| Cellulose acetate | 37.7 |
| Polaxamer F68 | 6.1 |
| Total weight of the rate controlling | 43.8 |
| membrane | |
| Immediate release overcoat | |
| salmeterol | 0.05 |
| fluticasone propionate | 0.05 |
| Hydroxypropylmethyl cellulose | 24.1 |
| Total weight of the immediate release | 24.2 |
| overcoat | |
| Total tablet weight | 270.55 |

### Example 5

### Treatment of Peptic Ulcer Disease

As seen in FIG. 1, it is very common for patients suffering from peptic ulcer disease to experience exacerbations around about midnight. A composition of famotidine (Table 6) is exemplified that will allow the patient to administer the tablet at bedtime but does not release the active drug until about 3-4 hours after administration.

A maximum peak plasma concentration resulting from the therapeutically effective dose may occur within about 1 hour to about 8 hours, alternatively about 1 hour to about 7 hours, alternatively about 1 hour to about 6 hours, alternatively about 1 hour to about 5 hours, alternatively about 1 hour to about 4 hours, alternatively about 1 hour to about 3 hours, alternatively about 1 hour to about 2 hours, after the administration of the dosage form. Alternatively, the maximum peak plasma concentration resulting from the therapeutically effective dose may occur at least about 30 minutes, alternatively about 1 hour, alternatively about 2 hours, alternatively about 3 hours, alternatively about 4 hours, alternatively about 5 hours, alternatively about 6 hours, alternatively about 7 hours, alternatively about 9 hours, alternatively about 9 hours, alternatively about 10 hours, alternatively about 11 hours, alternatively about 12 hours, after the administration of the dosage form.

The dosage form could be formulated such that there is a lag time of at least about 8 hours, alternatively about 7 hours, alternatively about 6 hours, alternatively about 5 hours, alternatively about 4 hours, alternatively about 3 hours, alternatively about 2 hours, alternatively about 1 hour, +/− about 30 minutes between administration/ingestion of the trilayer tablet and the time of the maximum plasma concentration (Tₘₐₓ) of the drug. Alternatively, the dosage form could be formulated such that there is a lag time of about 1 to about 6 hours, alternatively about 2 to about 5 hours, alternatively about 2 to about 4 hours, alternatively about 3 to about 4 hours, between administration/ingestion of the trilayer tablet and the time of the maximum plasma concentration (Tₘₐₓ) of the drug.

The delivery/release of the therapeutically effective dose of the active pharmaceutical ingredient or pharmaceutically acceptable salt thereof may result in the maximum plasma concentration (Cₘₐₓ, and Tₘ) occurring around about 12 AM, alternatively around about 1 AM, alternatively around about 1:30 AM, alternatively around about 2 AM, alternatively around about 10 PM, alternatively around about 10:30 PM, alternatively around about 11 PM, alternatively around about 11:30 PM, +/− about 30 minutes at night. The delivery/release of the therapeutically effective dose of the active pharmaceutical ingredient or pharmaceutically acceptable salt thereof may also result in the maximum plasma concentration (Cₘₐₓ, and Tₘₐₓ) occurring between about 9 PM and about 3 AM, alternatively between about 10 PM and about 2 AM, alternatively between about 11 PM and about 1 AM, alternatively between about 11:30 PM and about 12:30 AM, alternatively between about 11 PM and about 2 AM. (Note that all times expressed in this application would apply to the time zone where the patient resides and/or where the dose is administered.)

Drugs that could be delivered to treat peptic ulcer disease include but are not limited to famotidine, chlordiazepoxide, methscopolamine, propanthelene, clidinium, pantoprazole and glycopyrolate.

TABLE 6
Composition of Famotidine Trilayer Tablet

| | Amount per |
|---|---|
| Components | Tablet (mg) |
| First Layer | |
| Polyethylene oxide, 1 MM | 38.235 |
| Povidone (K29-32) | 1.25 |
| Croscarmellose sodium | 1.5 |
| Silicon Dioxide (Sylloid 244FP) | 1 |
| Sodium chloride | 7.5 |
| Magnesium Stearate | 0.25 |
| Butylated hydroxytoluene | 0.015 |
| Ferric oxide | 0.25 |
| Total weight of the first layer | 50 |
| Second Layer | |
| Famotidine | 40 |
| Polyethylene oxide, 100K | 41 |
| Povidone (K29-32) | 2.5 |
| Croscarmellose sodium | 5 |
| Silicon Dioxide (Sylloid 244FP) | 1 |
| Sodium chloride | 10 |
| Magnesium Stearate | 0.5 |
| Butylated hydroxytoluene | 0.025 |
| Ferric oxide | 0.025 |
| Total weight of the second layer | 100.05 |
| Push Layer | |
| Polyethylene oxide, 7000K | 130 |
| Povidone (K29-32) | 5 |
| Silicon Dioxide (Sylloid 244FP) | 1.33 |
| Sodium chloride | 13 |
| Magnesium Stearate | 0.5 |
| Butylated hydroxytoluene | 0.05 |
| Ferric oxide | 0.67 |
| Total weight of the push layer | 150.55 |
| Subcoat | |
| Hydroxypropylmethyl cellulose | 11.5 |
| Total weight of the subcoat | 11.5 |
| Rate controlling membrane | |
| Cellulose acetate | 37.7 |
| Polaxamer F68 | 6.1 |
| Total weight of the rate controlling | 43.8 |
| membrane | |
| Total tablet weight | 400 |

All publications, patent applications, and patents cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A pharmaceutical composition comprising a core, a semi-permeable membrane disposed generally around the core, an orifice in the semi-permeable membrane in fluid communication with the core, and a coating comprising a first therapeutically effective dose of an active pharmaceutical ingredient or a pharmaceutically acceptable salt thereof disposed generally around the semi-permeable membrane,
wherein the core comprises first, second, and third layers, wherein the first layer is in fluid communication with the orifice, wherein the second layer comprises a second therapeutically effective dose of the active pharmaceutical ingredient or a pharmaceutically acceptable salt thereof and is located adjacent to the first layer, and wherein the third layer is located adjacent the second layer, and
wherein the first layer has a higher viscosity than the second layer.

2. The composition of claim 1, wherein the first layer has a viscosity between about 50 and about 20,000 cps.

3. The composition of claim 1, wherein the first layer has a viscosity between about 50 and about 1500 cps.

4. The composition of claim 1, wherein the first layer has a viscosity between about 600 and about 1200 cps.

5. The composition of claim 1, wherein the first layer has a viscosity between about 500 and about 1000 cps.

6. The composition of claim 1, wherein the second layer has a viscosity between about 30 and about 10,000 cps.

7. The composition of claim 1, wherein the second layer has a viscosity between about 30 and about 1,000 cps.

8. The composition of claim 1, wherein the second layer has a viscosity between about 30 and about 100 cps.

9. The composition of claim 1, wherein the second layer has a viscosity between about 5 and about 50 cps.

10. The composition of claim 1, wherein the first layer comprises polyethylene oxide with a molecular weight of about 300K.

11. The composition of claim 1, wherein the first layer comprises polyethylene oxide with a molecular weight between about 200K to about 1000K.

12. The composition of claim 1, wherein the second layer comprises polyethylene oxide with a molecular weight of about 100K.

13. The composition of claim 1, wherein the second layer comprises polyethylene oxide with a molecular weight between about 100K to about 600K.

14. The composition of claim 1, wherein each of the first and second layers comprise polyethylene oxide having a molecular weight and wherein the molecular weight of the polyethylene oxide in the first layer is higher than the molecular weight of the polyethylene oxide in the second layer.

15. The composition of claim 1, wherein the active pharmaceutical ingredient is selected from the group consisting of carbamazepine, ethosuximide, oxcarbazepine, phenobarbital, phenytoin, primidone, topiramate, valproate, valproic acid, valproex sodium, felbamate, gabapentin, lamotrigine, levetiracetam, lacosamide, pregabalin, primidone, rufinamide, tiagabine and zonisamide.

16. The composition of claim 1, wherein the active pharmaceutical ingredient is selected from the group consisting of alprazolam, bretazenil, bromazepam, chlordiazepoxide, cinolazepam, clonazepam, cloxazolam, clorazepate, diazepam, estazolam, fludiazepam, flunitrazepam, flurazepam, halazepam, flutoprazepam, ketazolam, loprazolam, lorazepam, lormetazepam, medazepam, nitrazepam, midazolam, nordazepam, oxazepam, phenazepam, pinazepam, prazepam, quezepam, temazepam, tetrazepam, and triazolam.

17-44. (canceled)

45. A method for treating epilepsy, comprising the steps of:
providing a dosage form comprising a core, a semi-permeable membrane disposed generally around the core, an orifice in the semi-permeable membrane in fluid communication with the core, and a coating comprising a first therapeutically effective dose of a first active pharmaceutical ingredient or a pharmaceutically acceptable salt thereof disposed generally around the semi-permeable membrane; and
administering the dosage form to a patient suffering from epilepsy,
wherein the core comprises first, second, and third layers, wherein the first layer is in fluid communication with the orifice, wherein the second layer comprises a second therapeutically effective dose of a second active pharmaceutical ingredient or pharmaceutically acceptable salt thereof and is located adjacent to the first layer, and wherein the third layer is located adjacent the second layer, and
wherein a maximum peak plasma concentration resulting from the second therapeutically effective dose occurs within about 5 hours to about 11 hours after the occurrence of a maximum peak plasma concentration resulting from the first therapeutically effective dose.

46-72. (canceled)

73. A method for treating a psychiatric disorder, comprising the steps of:
providing a dosage form comprising a core, a semi-permeable membrane disposed generally around the core, an orifice in the semi-permeable membrane in fluid communication with the core, and a coating comprising a first therapeutically effective dose of a first active pharmaceutical ingredient or a pharmaceutically acceptable salt thereof disposed generally around the semi-permeable membrane; and
administering the dosage form to a patient suffering from the psychiatric disorder,
wherein the core comprises first, second, and third layers, wherein the first layer is in fluid communication with the orifice, wherein the second layer comprises a second therapeutically effective dose of a second active pharmaceutical ingredient or pharmaceutically acceptable salt thereof and is located adjacent to the first layer, and wherein the third layer is located adjacent the second layer, and
wherein a maximum peak plasma concentration resulting from the second therapeutically effective dose occurs within about 5 hours to about 14 hours after the occurrence of a maximum peak plasma concentration resulting from the first therapeutically effective dose.

74-100. (canceled)

101. A method for treating asthma, comprising the steps of:
providing a dosage form comprising a core, a semi-permeable membrane disposed generally around the core, and an orifice in the semi-permeable membrane in fluid communication with the core; and
administering the dosage form to a patient suffering from asthma,
wherein the core comprises first, second, and third layers, wherein the first layer is in fluid communication with the orifice, wherein the second layer comprises a therapeutically effective dose of an active pharmaceutical ingredient or pharmaceutically acceptable salt thereof and is located adjacent to the first layer, and wherein the third layer is located adjacent the second layer,
wherein a maximum peak plasma concentration resulting from the therapeutically effective dose occurs at least about 5 to about 11 hours after administration of the dosage form.

102-166. (canceled)

167. A method for treating peptic ulcer disease, comprising the steps of:
providing a dosage form comprising a core, a semi-permeable membrane disposed generally around the core, and an orifice in the semi-permeable membrane in fluid communication with the core; and
administering the dosage form to a patient suffering from peptic ulcer disease,
wherein the core comprises first, second, and third layers, wherein the first layer is in fluid communication with the orifice, wherein the second layer comprises a therapeutically effective dose of an active pharmaceutical ingredient or pharmaceutically acceptable salt thereof and is located adjacent to the first layer, and wherein the third layer is located adjacent the second layer,
wherein a maximum peak plasma concentration resulting from the therapeutically effective dose occurs at least about 1 hour after administration of the dosage form.

168-239. (canceled)
